(19) United States
(12) Plant Patent Application Publication
(10) Pub. No.: US 2018/0220568 P1
(43) Pub. Date: Aug. 2, 2018

(54) Raspberry plant named 'PS-08.095-01'

(50) ;

(71) Applicant: Plant Sciences, Inc., Watsonville, CA (US)

(72) Inventor: Scott W. Adams, Watsonville, CA (US)

(73) Assignee: Plant Sciences, Inc., Watsonville, CA (US)

(21) Appl. No.: 15/932,044

(22) Filed: Jan. 29, 2018

Related U.S. Application Data
(60) Provisional application No. 62/453,361, filed on Feb. 1, 2017.

(51) Int. Cl. A01H 6/74

(52) U.S. Cl. PLT/204

(57) ABSTRACT

This invention relates to a new and distinct everbearing variety of raspberry plant named ‘PS-08.095-01’. The new variety is primarily adapted to the growing conditions of the central coast of California and is characterized by the following: early mid-season primocane production with medium to large sized fruit of medium to dark-red coloration. Fruit is of consistent blunt conic shape, releases with some difficulty from receptacle, possessing large drupelets and is of good flavor. Foliage is strongly concave, medium green; possessing medium rugosity and mostly 3 foliate. Primocanes have a strong waxy coat, purple spines, fairly high thorn density and have weak to medium anthocyanin coloration.

Latin name of the genus and species of the plant claimed: Rubus idaeus.

Variety denomination: ‘PS-08.095-01’.

### BACKGROUND OF THE INVENTION

The present invention relates to a new and distinct fall bearing raspberry variety designated as ‘PS-08.095-01’. This new variety is a result of a controlled cross made in 2008 in Watsonville, Calif. between raspberry variety ‘04.3942’ (unpatented) as the female parent and raspberry variety ‘GRANDEUR’ (patented, U.S. Plant Pat. No. 20,459) as the male parent in an ongoing breeding program. The variety is botanically known as Rubus idaeus.

The seedling resulting from the aforementioned cross was asexually propagated by dormant canes in Santa Cruz County, Calif. and was subsequently selected by the inventor from a controlled breeding plot in Watsonville, Calif. in 2010. After its selection, the new variety was further asexually propagated by dormant canes, roots and non-dormant root shoot cuttings in Santa Cruz County, Calif., San Joaquin County, Calif. and Siskiyou County, Calif. The new variety was then extensively tested over the next several years in fruiting fields in Santa Cruz County, Calif. This propagation has demonstrated that the combination of traits disclosed herein as characterizing the new variety are fixed and remain true to type through successive generations of asexual reproduction.

BRIEF SUMMARY OF THE INVENTION

‘PS-08.095-01’ is primarily adapted to the climate and growing conditions of the central coast of California. This region provides the necessary year-round temperatures required for it to produce and maintain a strong vigorous plant and to remain in fruit production from July through December on primocanes and in the ensuing year from May through July on floricanes. The following traits have been repeatedly observed and are determined to be unique characteristics of ‘PS-08.095-01’, which in combination distinguish this raspberry plant as a new and distinct variety:
1. Dark purple spines
2. High fruit yield
3. Medium to dark red fruit color
4. Strong primocane glaucosity
5. Good flavor

The raspberry variety that is believed to be most closely related to the new raspberry variety ‘PS-08.095-01’ is the raspberry variety ‘GRANDEUR’ (patented, U.S. Plant Pat. No. 20,459). In comparison to the similar raspberry variety ‘GRANDEUR’, ‘PS-08.095-01’ differs by the following combination of characteristics described in Table 1:

TABLE 1

| | | ‘GRANDEUR’ |
|---|---|---|
| Characteristic | ‘PS-08.095-01’ | (US PP20,459) |
| Primocane waxy coat | Strong | Absent to very weak |
| (glaucosity) | | |
| Predominate number | Mostly 3 | Always 3 |
| of leaflets | | |
| Leaf shape (cross section) | Strongly concave | Flat to slightly convex |
| Thorn density per cm | 8.53 | 3.38 |
| at central 1/3 | Dense | Medium |
| Thorn coloration (tip) | RHS N186B | RHS 180C |
| | Greyed-purple group | Greyed-red group |
| Thorn coloration (base) | RHS N186C | RHS 145B |
| | Greyed-purple group | Yellow-green group |
| Color mature fruit | RHS 46A | RHS 42A |
| | Medium to dark red | Medium red |

‘PS-08.095-01’ differs from its parents, ‘GRANDEUR’ and ‘04.3942’, by the following combination of characteristics described in Table 2:

TABLE 2

| | | ‘GRANDEUR’ | |
|---|---|---|---|
| Characteristic | ‘PS-08.095-01’ | (US PP15,439) | ‘04.3942’ |
| Productivity | High | High | Medium |
| Glossiness (fruit) | Medium | High | Medium |
| Firmness (fruit) | Medium to high | High | Low |
| Adherence of receptacle | Weak to medium | Weak | Medium |
| Primocane time of fruiting | Medium | Medium | Medium |
| Fruit size | Medium to large | Medium | Large |
| Flavor | Good | Good | Excellent |

For identification, a series of molecular markers have been determined for this new variety.

### BRIEF DESCRIPTIONS OF THE DRAWINGS

The accompanying color photographs illustrate the overall appearance of typical specimens of the new raspberry variety ‘PS-08.095-01’ at various stages of development as true as reasonably possible with color reproductions of this type. Color in the photographs may differ slightly from the color value cited in the detailed botanical description which accurately describes the color of ‘PS-08.095-01’. The depicted plant and plant parts of the new raspberry variety ‘PS-08.095-01’ were taken in Watsonville, Calif. and are approximately 2 to 16 months old:

FIG. 1 shows typical primocane foliage and fruit color; foliate and rugosity characteristics of ‘PS-08.095-01’ taken in the month of May 2015;

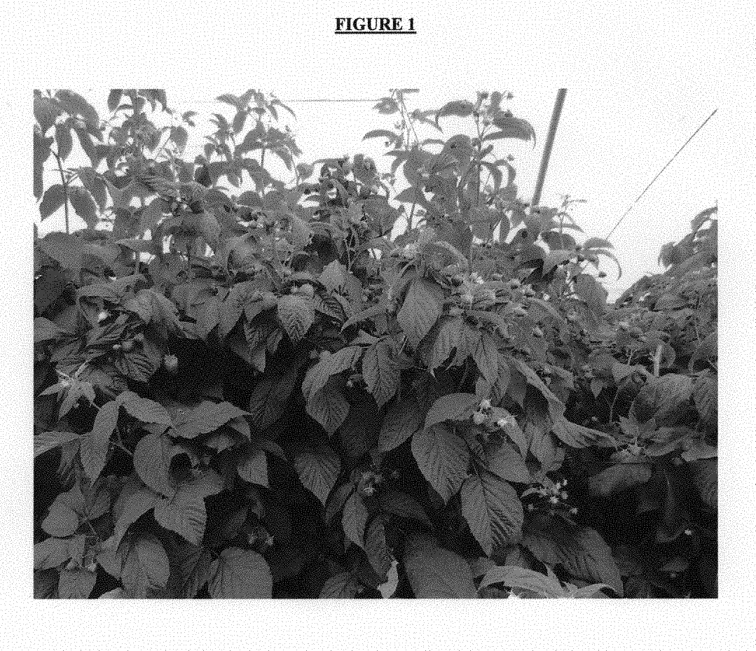

FIG. 2 shows typical gloss, coloration, and drupelet formation of floricane fruit of ‘PS-08.095-01’ taken in the month of May 2015;

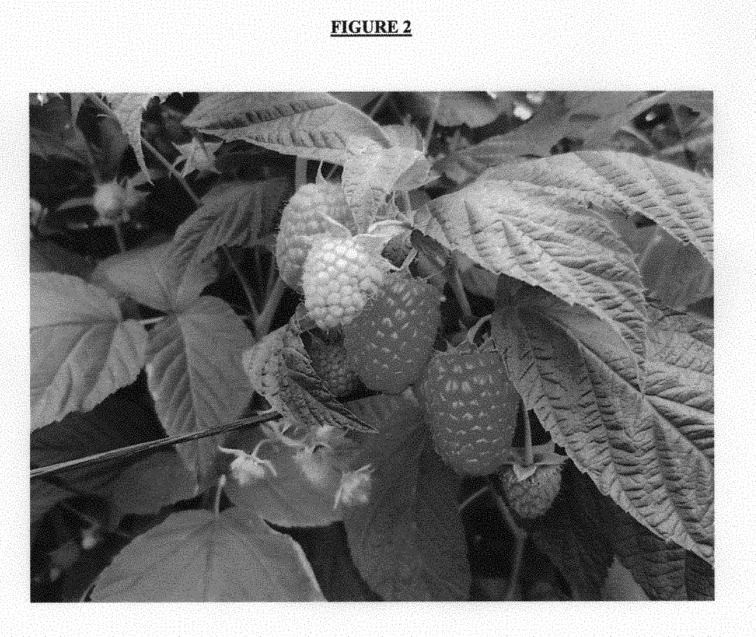

FIG. 3 shows typical harvested fruit of ‘PS-08.095-01’ taken in the month of July 2015;

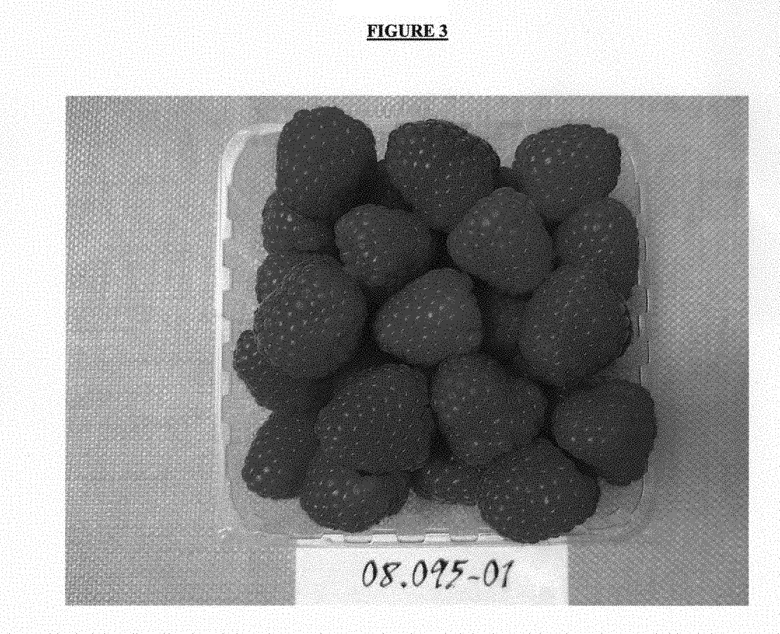

FIG. 4 shows typical dormant cane color characteristics of ‘PS-08.095-01’ taken in the month of January 2013.

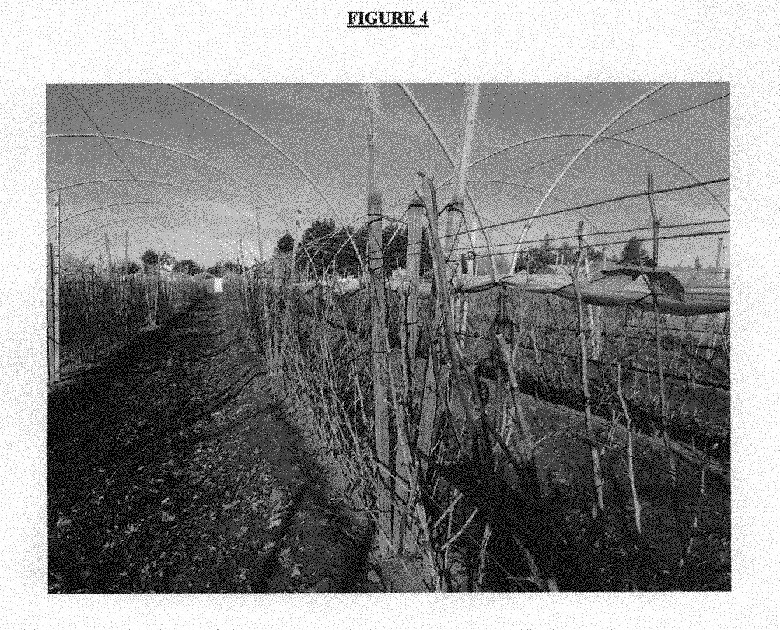

### DETAILED BOTANICAL DESCRIPTION

‘PS-08.095-01’ has not been observed under all possible environmental conditions. The characteristics of the new variety may vary in detail, depending upon variations in environmental factors, including weather (temperature, humidity and light intensity), day length, soil type, and location.

The aforementioned photographs, together with the following description of the new raspberry variety ‘PS-08.095-01’, unless otherwise noted, are based upon observations taken during the 2016-2017 growing season in Watsonville, Calif. Primocane measurements and ratings were taken from plants of ‘PS-08.095-01’ dug from a nursery located in Siskiyou County, Calif. during the middle of October 2015 and planted approximately 3 to 4 weeks later in Watsonville, Calif. The approximate age of the observed primocane plants is 7 to 8 months. Floricane measurements and ratings were taken from the same planting of ‘PS-08.095-01’ at an approximate age of 16 to 18 months. Yield observations and fruit quality characteristics are averaged from three years of data collected from the 2013 through 2016 production seasons. Flower measurements and characteristics are from secondary flowers unless otherwise noted. Fruit characteristics and measurements are from secondary fruit unless otherwise noted. Foliage characteristics and measurements arc from 3-foliate foliage unless otherwise noted.

Color terminology where noted follows The RHS Colour Chart Fifth Edition, Royal Horticultural Society, London, United Kingdom (1966).

The following Tables 3-7 describe fruit, plant, foliage, flower and pest/disease characteristics of the new raspberry ‘PS-08.095-01’ in comparison to the similar raspberry variety ‘GRANDEUR’ (patented, U.S. Plant Pat. No. 20,459).

TABLE 3
FRUIT CHARACTERISTICS

| | | ‘GRANDEUR’ |
|---|---|---|
| Characteristic | ‘PS-08.095-01’ | (US PP20,459) |
| Color mature fruit | RHS 46A | RHS 42A |
| | Medium to dark red | Medium red |
| Color achenes | RHS 159A | RHS 159A |
| | Orange-white group | Orange-white group |
| Fruit length (mm) | 25.52 | 22.91 |
| Fruit width (mm) | 23.13 | 20.12 |
| Length/Width ratio | 1.10 | 1.14 |
| | Longer than broad | Longer than broad |
| Seed weight (mg) | 1.77 | 1.75 |
| Drupelets per berry | 105 | 93 |
| Weight of single drupe | 0.061 | 0.043 |
| (g/drupe) | | |
| Relative size of drupes | Large | Medium |
| Fruit size | Medium to large | Medium |
| Predominant shape | Conical | Conical |
| Evenness of color | Even | Even |
| Glossiness | Medium | Medium |
| Adherence of receptacle | Weak to medium | Very weak |
| Firmness of flesh | Firm | Very firm |
| Firmness of skin | Firm | Very firm |
| Soluble solids (% Brix) | 10.2 | 10.5 |
| Flavor | Good | Good |

TABLE 4
PLANT CHARACTERISTICS

| | | ‘GRANDEUR’ |
|---|---|---|
| Characteristic | ‘PS-08.095-01’ | (US PP20,459) |
| General: | | |
| Habit | Upright | Upright |
| Size | Medium | Medium |
| Productivity | High | High |
| Self-fruitfulness | Yes | Yes |
| Type of bearing | Everbearing | Everbearing |
| Primocane: | | |
| Color (true) | RHS 145B | RHS 145B |
| | Yellow-green group | Yellow-green group |
| Length (cm) | 162.7 | 143.6 |
| Basal diameter (mm) | 16.33 | 21.80 |
| Diameter central 1/3 (mm) | 11.39 | 12.55 |
| Lateral length at | 43.4 | 43.6 |
| central 1/3 (cm) | | |
| No. fruiting laterals per cane | 12.8 | 17.4 |
| Internode length at | 36.51 | 48.84 |
| central 1/3 (mm) | | |
| Anthocyanin coloration | RHS 59B | RHS 59B |
| | Red-purple group | Red-purple group |
| Anthocyanin intensity | Weak to medium | Weak |
| Pubescence | Absent | Absent |
| Length of vegetative | 7.29 | 11.01 |
| bud (mm) | | |
| Strength of waxy coat | Strong | Absent to very weak |
| (glaucosity) | | |
| Time of flowering | Medium | Medium |
| Time of fruiting | Medium | Medium |
| Length of fruiting season | Long | Long |
| % of total yield | 55% | 50% |
| Flowering period | Late June to | Late June to |
| | Late November | Late November |
| Harvest period | Late July to | Late July to |
| | Mid December | Mid December |
| Primocane fruit weight (g) | 6.1 | 3.8 |
| Primocane yield (g/plant) | 3,721 | 2,662 |
| Young Shoots: | | |
| Number (per meter) | 15-20 | 15-20 |
| | Medium | Medium |
| Anthocyanin presence | Present | Present |
| Anthocyanin coloration | RHS 179B | RHS 179A |
| | Red-purple group | Greyed-red group |
| Anthocyanin intensity | Weak | Medium |
| Thorns: | | |
| Thorn coloration (tip) | RHS N186B | RHS 180C |
| | Greyed-purple group | Greyed-red group |
| Thorn coloration (base) | RHS N186C | RHS 145B |
| | Greyed-purple group | Yellow-green gioup |
| Thorn length at | 0.88 | 0.76 |
| central 1/3 (mm) | | |
| Thorn base at | 0.75 | 1.53 |
| central 1/3 (mm) | | |
| Thorn presence | Present | Present |
| Thorn density per cm | 8.53 | 3.38 |
| at central 1/3 | Dense | Medium |
| Thorn texture | Rigid | Rigid |
| Attitude of the tip | Horizontal | Horizontal |
| Floricane: | | |
| Color (true) | RHS 164A | RHS 164A |
| | Greyed-orange group | Greyed-orange group |
| Length (cm) | 121.3 | 119.8 |
| Fruiting lateral attitude | Erect | Erect |
| Time bud burst | Medium | Medium |
| Time of flowering | Medium | Medium |
| Time of fruiting | Medium | Medium |
| Length of fruiting season | Medium to long | Medium to long |
| % of total yield | 45% | 50% |
| Flowering period | Late April to | Late April to |
| | Late June | Late June |
| Harvest period | Late May to | Late May to |
| | Late July | Late July |
| Floricane fruit weight (g) | 5.9 | 3.7 |
| Floricane yield (g/plant) | 2,941 | 2,680 |

TABLE 5
FOLIAGE CHARACTERISTICS

| | | ‘GRANDEUR’ |
|---|---|---|
| | ‘PS-08.095-01’ | (US PP20,459) |
| Characteristic | (3 Foliate) | (3 Foliate) |
| General: | | |
| Color of upper surface | RHS 139A | RHS N137A |
| | Green group | Green group |
| Color of lower surface | RHS 190B | RHS 190B |
| | Greyed-green group | Greyed-green group |
| Shape in cross section | Strongly concave | Flat to slightly convex |
| Arrangement | Compound | Compound |
| Relief between veins | Medium | Strong |
| (rugosity) | | |
| Glossiness | Medium | Weak |
| Number of leaflets/leaf | Mostly 3 | Always 3 |
| Terminal Leaflet: | | |
| Length (mm) | 136.2 | 127.5 |
| Width (mm) | 100.5 | 91.6 |
| Length/width ratio | 1.3 | 1.4 |
| | Longer than broad | Longer than broad |
| Size | Medium | Medium to large |
| Shape | Cordate | Cordate |
| Shape of base | Cordate | Cordate |
| Shape of tip | Acuminate | Acuminate |
| Margins | Biserrate | Biserrate |
| Lateral Leaflet: | | |
| Length (mm) | 114.6 | 105.5 |
| Width (mm) | 76.5 | 65.1 |
| Length/width ratio | 1.5 | 1.6 |
| | Longer than broad | Longer than broad |
| Rachis length (mm) | 32.7 | 36.2 |
| Orientation | Opposite | Opposite |
| Arrangement | Compound | Compound |
| Shape | Ovate | Ovate |
| Overlapping | Touching | Touching |
| Shape of the base | Rounded | Oblique rounded |
| Shape of the tip | Acuminate | Acuminate |
| Margins | Biserrate | Biserrate |
| Petiole: | | |
| Length (mm) | 48.60 | 59.00 |
| Width (mm) | 3.35 | 3.99 |
| Thorn presence | Yes | Yes |
| Thorn orientation | Erect | Erect |
| Anthocyanin coloration | RHS 186C | RHS 184C |
| of upper surface | Greyed-purple group | Greyed-purple group |
| Anthocyanin intensity of | Absent to very weak | Weak |
| upper surface | | |
| Stipule length (mm) | 16.61 | 10.23 |
| Stipule orientation | Erect | Erect to horizontal |

TABLE 6
FLOWER CHARACTERISTICS

| | | ‘GRANDEUR’ |
|---|---|---|
| Characteristic | ‘PS-08.095-01’ | (US PP20,459) |
| Petal color | 155C | 155C |
| | White group | White group |
| Flower diameter (mm) | 22.70 | 22.55 |
| Petal Length (mm) | 7.44 | 6.42 |
| Petal width (mm) | 3.40 | 3.11 |
| Petal length/width ratio | 2.19 | 2.06 |
| | Much longer | Much longer |
| | than broad | than broad |
| No. petals/flower | 5.1 | 5.2 |
| No. sepals/flower | 5.1 | 5.2 |
| Relative number of | 19.2 | 16.2 |
| pedicel thorns | Medium | Medium |
| Peduncle anthocyanin presence | Present | Present |
| Peduncle anthocyanin | RHS 184B | RHS 184A |
| coloration | Greyed-purple group | Greyed-purple group |
| Peduncle anthocyanin intensity | Strong | Medium |

TABLE 7
PEST AND DISEASE REACTIONS

| | | ‘GRANDEUR’ |
|---|---|---|
| Characteristic | ‘PS-08.095-01’ | (US PP20,459) |
| Spotted wing drosophila | Susceptible | Susceptible |
| (Drosophila suzukii) | | |
| Two-spotted spider mite | Susceptible | Susceptible |
| (Tetranychus urticae) | | |
| Grey fruit mold | Susceptible | Susceptible |
| (Botrytis cinerea) | | |
| Powdery mildew | Moderately resistant | Moderately susceptible |
| (Podosphaera aphanis | | |
| var. aphanis) | | |
| Yellow rust | Moderately resistant | Moderately susceptible |
| (Phragmidium rubi-idaei) | | |

We claim:

1. A new and distinct raspberry variety named ‘PS-08.095-01’, as herein described and illustrated by the characteristics set forth above.
